(19) United States
(12) Patent Application Publication
(10) Pub. No.: US 2008/0314750 A1
(43) Pub. Date: Dec. 25, 2008

(54) Method for Pre-Fractionation of Complex Samples

(75) Inventors: Asa Hagner-McWhirter, Uppsala (SE); Jesper Hedberg, Uppsala (SE)

Correspondence Address: GE HEALTHCARE BIO-SCIENCES CORP.;PATENT DEPARTMENT, 800 CENTENNIAL AVENUE, PISCATAWAY, NJ, 08855, US

(73) Assignee: GE HEALTHCARE BIO-SCIENCES AB, UPPSALA (SE)

(21) Appl. No.: 12/093,158

(22) PCT Filed: Nov. 14, 2006
(86) PCT No.: PCT/SE06/01286

§ 371 Date: May 9, 2008

(30) Foreign Application Priority Data
Nov. 18, 2005 (SE) 0502552-3

(51) Int. Cl. C07K 1/28; C07K 1/36

(52) U.S. Cl. 204/459; 204/610; 530/416

(57) ABSTRACT

The present invention relates to a method for pre-fractionation of complex protein and/or peptide samples. The method comprises the following steps:
a) loading the sample onto pre-packed media able to separate proteins/peptides according to their pl-value;
b) eluting the media by centrifugal force under denaturing conditions with at least three buffers having different pH in a stepwise manner to obtain at least three fractions separated according to pl-value; and
c) subjecting each fraction to further separation in a pH-range corresponding to the pl-values of said fractions.

The invention also relates to a kit comprising at least one spin column or multiwell plate filled with chromatography medium able to separate proteins/peptides according to pl-value, at least three buffers, and one or more IPG strips having a narrow pH-range adapted to each of said buffers.

### CROSS-REFERENCE TO RELATED APPLICATIONS

This application is a filing under 35 U.S.C. § 371 and claims priority to international patent application number PCT/SE2006/001286 filed Nov. 14, 2006, published on May 24, 2007, as WO 2007/058584, which claims priority to patent application number 0502552-3 filed in Sweden on Nov. 18, 2005.

### FIELD OF THE INVENTION

The present invention relates to a method for pre-fractionation of complex protein and peptide samples. In the method the sample is first subjected to a liquid phase separation according to pl as a pre-fractionation before further protein analysis, preferably by 2D electrophoresis.

### BACKGROUND OF THE INVENTION

Proteomic separation techniques such as isoelectric focusing (IEF) and multi-dimensional liquid chromatography (MDLC) have a wide spread use. Generally electrophoretic techniques like IEF and sodium dodecyl (SDS) electrophoresis give, when used at the protein level in gel, much better resolution and protein yields than chromatographic alternatives. Two-dimensional, 2-D, electrophoresis based on the combination of these two techniques, IEF and SDS electrophoresis, is also a commonly used approach when separation of very complex samples is conducted at the protein level.

Sample preparation methods and devices for pre-treatment or pre-fractionation of samples have been identified as important for future proteomics applications, especially for complex samples. For two-dimensional (2D) application, such as 2D electrophoresis, and as well other proteomics methods there is a dare need for fractionating complex protein samples, such as plasma samples, to improve throughput and out-put data. Complex samples contain a large number of proteins some of which are high abundant and some of which are low abundant. It is most often desired to detect and analyse the low abundant or rare proteins in a complex sample. However, this often requires the sample to be sub-divided or pre-fractionated before further separation, such as 2D electrophoresis.

One problem with 2D electrophoresis using for instance narrow range pH strips covering a limited pH range is that when applying a crude or non-fractionated sample, a large portion of the proteins display pl's outside of the particular pH range of the strip. These proteins will during focusing migrate out of the narrow range strip ending up at the cathode or anode electrode. Hence, only a smaller portion of the proteins in a crude sample will be analysed when using a narrow range strip. A method for sub-dividing a protein sample is therefore needed.

Several preparative pre-fractionation procedures have been suggested to overcome this problem. For example, a fractionation kit is commercially available, 2D Fractionation Kit, that divides samples into fractions according to their ability to precipitate under certain conditions. Unfortunately, this kit contains many steps and is time consuming. Also, SEPHADEX™ isoelectric focusing has been suggested as a pre-fractionation procedure before 2D separation (Görg, A., et. al., Sample prefractionation with SEPHADEX™ isoelectric focusing prior to narrow pH range two-dimensional gels. 2002, 2, 1652-1657, Proteomics). Other isoelectrofocusing based pre-fractionating techniques have been investigated. Sample pre-fractionation was obtained via multi-compartment electrolyzers with isoelectric membranes (Herbert and Righetti, A turning point in proteomics: sample prefractionation via multicompartment electrolyzers with isoelectric membranes. 2000, 21, 3639-3648, Electrophoresis) and by using a novel solution isoelecrofocusing device (Zuo and Speicher, A method for global analysis of complex proteomes using sample pre-fractionation by solution isoelectrofocusing prior to two-dimensional electrophoresis. 2000, 284, 266-278, Anal. Biochem.)

Ion exchange as a pre-fractionation step before 2D gel electrophoresis has also been tried (Butt, A. et al., Chromatographic separations as a prelude to two-dimensional electrophoresis in proteomics analysis. Proteomics 2001, 1, 42-53), but little correlation between theoretical and practical pl and elution position on ion exchange using non-denaturating conditions was found.

However, the above methods and in particular SEPHADEX™ isoelectric focusing is very tedious and labour intensive. Therefore, there is a need of a simple and rapid pre-fractionation or pre-treatment method, especially for detection of low abundant and/or rare proteins in complex samples.

### SUMMARY OF THE INVENTION

This invention provides a simple and rapid solution for reduction of complexity of protein and/or peptide samples. According to the invention a mixed protein or peptide sample is pre-fractionated according to pl by ion exchange chromatography or chromatofocusing by using centrifugal force. The sample is first bound to a pre-packed media (preferably spin column format or multiwell format) at high pH or low pH depending on the matrix used (˜pH 11 for anion exchange columns and ˜pH 3 for cation exchange columns). For the situation with an anion exchange column, proteins with pl below 11 will then be bound to the column and the proteins with pl above 11 will elute in the flow through. Most proteins and peptides have a pl below 11 and will therefore bind to the column. The flow through will probably contain no or very few proteins/peptides. The proteins/peptides in the column are subsequently eluted during centrifugation in a step wise manner by lowering the pl of the elution buffer. The protein/peptide will be eluted at its pl (no charge) or just below its pl (+-charged). At least 3 different elution buffers can be used to create at least 4 pl fractions including the flow through fraction of the protein/peptide sample and the complexity of the sample is thereby significantly reduced.

Thus, the present inventors provide a convenient and rapid methodology to sub-divide a protein and/or peptide sample by separating the proteins/peptides into fractions based on charge and thereby pl-value. Thereby a larger part of the proteins/peptides in a sample can be analysed when IPG strips and protein/peptide fractions with matching pH ranges and pl's, respectively, are used.

According to the invention all of the proteins/peptides loaded on the strips will focus within the strip's pH range. A pl fractionated sample will make it possible to load higher concentration of the proteins/peptides of interest i.e., proteins/peptides that will focus on a narrow range IPG strip (no cathodic and anodic losses), which will result in increased possibilities for analysis and identification in the 2 D electrophoresis workflow. This leads to higher intensity spot maps and a significant increase of the possibility to detect for instance low abundance proteins/peptides.

One important feature of the present invention is that it mimics standard IEF conditions by using the same denaturing conditions in the starting and elution buffers, which will result in a higher correlation between pl elution in the chromatofocusing/ion exchange step and migration position on the IPG strip.

All fractions are then further analysed, for example the fractions may be applied on a narrow range IPG (immoblised pH gradient) strip or gel resulting in improved effective loading capacity compared to non-fractionated samples because the present invention enables application of a higher concentration of proteins/peptides that will focus on a narrow range IPG strip or gel. Without a pre-fractionation a large part of the sample will, for instance, on a narrow range IPG strip migrate out of the strip and end up in the anode or cathode electrode, since the pl's of many proteins/peptides differs significantly from what is covered in the pH gradient of the strip. Therefore, a higher spot intensity will be achieved by applying a fractionated sample only containing proteins/peptides with a roughly defined pl value.

If necessary, the method also includes a buffer exchange step (also in spin column format or multiwell format) enabling direct application of the fractions to further separation, other than 2D electrophoresis.

Thus, in a first aspect the invention provides a method for pre-fractionation or sub-division of a complex sample, i.e. a sample containing a large number of proteins and/or peptides, comprising the following steps:
a) loading the sample onto pre-packed media able to separate proteins according to their pl-value;
b) eluting the sample by centrifugal force under denaturing conditions with at least three buffers having different pH in a stepwise manner to obtain at least three fractions separated according to pl-value; and
c) subjecting each fraction to further separation in a pH-range corresponding to the pl-values of said fractions.

In a preferred embodiment the flow through is collected before step b), in this way all fractions representing the total sample are obtained.

The complex sample may be of any origin such as, human or animal body fluid, such as plasma, human or animal tissue, mammalian cells, plant samples, bacterial or yeast cell samples.

The pre-packed media is preferably a chromatofocusing or ion exchange media.

Preferably, the ion exchange media in step a) is in a spin column or a multiwell plate format, preferably a 96 well filter plate.

If the starting buffer has pH 11, the flow through will contain proteins/peptides with pl >11. Preferably, the three elution buffers in step b) have pH 7-9, 6-4 and 3-2, respectively. More preferably, the buffers have pH 8, 5 and 3, respectively. The pH 8 buffer will elute proteins/peptides with pl: 11-8, the pH 5 buffer will elute proteins/peptides with pl 8-5, and the pH 3 buffer will elute proteins/peptides with pl 5-3.

Preferably, the spin column or multiwell plate is filled with a chromatofocusing matrix or ion exchange matrix for instance a mixed ion exchange media, a weak anion exchange media, a weak cation exchange media, a strong anion exchange media or a strong cation exchange media. Examples are MINIBEADS™, MONOBEADS™, SOURCE™ 15, SOURCE™ 30, SEPHAROSE™ HIGH PERFORMANCE, CAPTO™, SEPHAROSE™ FAST FLOW, SEPHAROSE™ XL, MONO P™, PBE 94, PBE 118, SOURCE S™, SOURCE Q™, DEAE, UNOSPHERE™, MACRO-PREP HIGH Q™, MACRO-PREP HIGH S™, CM™, AG MEDIA™, BIO-REX MEDIA™ and CHELEX MEDIA™.

Preferably a weak anion exchanger such as MONO P™ or DEAE. Alternatively, mixes of the above chromatography media may be used.

In an alternative embodiment, the spin column or multiwell plate is eluted with at least 5 buffers having pH 9, pH 7, pH 6, pH 5 and pH 3, this elutes proteins/peptides having pl of 11-9, 9-7, 7-6, 6-5, 5-3. If the starting buffer has a pH of 11, the flow through will contain proteins/peptides with pl above 11 (no, or very few proteins/peptides).

The further separation in step c) is preferably 2D electrophoresis.

The fractionated samples can directly be applied onto 2D electrophoresis.

In one embodiment the separation in step c) is electrophoretic separation on a narrow range IPG (immobilised pH gradient) strip or gel, such as pH 7-11, pH 6-9, pH 3-5.6 or other suitable IPG strips depending on proteins/peptides of interest with pH ranges 3-5.6, 5.3-6.5, 6.2-7.5, 3.5-4.5, 4-5, 4.5-5.5, 5-6, 5.5-6.7, 3-7, 4-7, 7-11 or 6-9.

In a second aspect, the invention relates to a kit comprising at least one spin column or multiwell plate filled with chromatography medium able to separate proteins and/or peptides according to pl-value, at least three buffers, and one or more IPG strips having a narrow pH-range adapted to each of said buffers.

The chromatography medium is an ion exchange medium or chromatofocusing medium. Preferred medias are MONO P™ or DEAE.

Preferably, the chromatography medium is an anion exchange or chromatofocusing medium and preferably the three buffers have pH values in the intervals pH 7-9, 6-4 and 3-2, respectively. The three buffers may be selected from 7M Urea, 2M Thiourea, 0.5% CHAPS™, 0.5% DTT and 0.5-2% ampholyte, tris pH 8 or polybuffer pH 5 or glycine pH 3.

Alternatively, the kit comprises at least five buffers. In this case, the five buffers preferably have pH 9, pH 7, pH 6, pH 5 and pH 3, respectively. The five buffers may be selected from 7M Urea, 2M Thiourea, 0.5% CHAPS™, 0.5% DTT and 0.5-2% ampholyte, tris pH 8 or tris pH 7 or polybuffer pH 6 or polybuffer pH 5 or glycine pH 3.

In one embodiment, the IPG strips are pH 7-11, pH 6-9 and 3-5.6 NL combined with the above three buffers.

In another embodiment, the IPG strips are pH 7-11 (for both elution fraction 1 and 2), pH 6-9, pH 5.3-6.5 and pH 3-5.6 NL combined with the above five buffers.

### DETAILED DESCRIPTION OF THE INVENTION

The invention will now be described in association with some non-limiting examples.

### Pre-Treatment

### a) Preparation of Anion Column

300 μl anion exchange media (MONO P™) is packed in a spin column with a filter on the top of the media. The column is equilibrated with 5 bed volumes basic starting buffer; Tris buffer pH 11.

All buffers also contain 7M Urea, 2M Thiourea, 0.5% CHAPS™, 0.5% DTT and 0.5-2% ampholyte to obtain denaturing conditions.

### b) Sample Application and Elution

500 μg CHO (Chinese hamster ovary) cell lysate (+ the same volume 2× glycine-NaOH buffer pH 11 buffer) is applied to the syringe/column.

The sample is serially eluted from the column with 300 μl tris buffer pH 8, thereafter with 300 μl polybuffer 74 buffer pH 5, and finally with 300 μl glycine-HCl buffer pH 3.

### c) Centrifugation

The elution is performed under centrifugal force. The spin column is placed in an empty centrifugation tube for each fraction and centrifuged after applying the sample and the different elution buffers.
Four fractions are obtained:
1. Flow through, pl>11
2. Elution 1, pl 8-11
3. Elution 2, pl 5-8
4. Elution 3, pl 3-5

The protein concentration in each fraction is determined using, for example, the 2D QUANT™ kit.

### 2D Analysis

In the sections below, a) and b) represent an experimental design to confirm the recovery and distribution of the proteins in the various fractions. c) represents an experimental design where only one or a few of the fractions are analysed in order to investigate the proteins in the individual fractions.

### a) Labelling

50 μg of protein from each fraction is labelled with:
Fraction 1: CY3™ DIGE Fluor minimal dye
Fraction 2: CY5™ DIGE Fluor minimal dye
Fraction 3: CY3™ DIGE Fluor minimal dye
Fraction 4: CY5™ DIGE Fluor minimal dye Non-fractionated sample, 2×50 μg, is labelled with CY2™ DIGE Fluor minimal dye.

### b) 2D Electrophoresis Using Wide Range IPG Strips

Two 24 cm long pH 3-11NL IPG Strips are prepared and two 26 cm×20 cm, 12% polyacrylamide gels are cast.

25 μg (from each sample) of fraction 1 and 3+CY2™ labelled non-fractionated sample are applied on a first IPG strip.

25 μg (from each sample) of fraction 2 and 4+CY2™ non-fractionated sample are applied on a second IPG strip. After isoelectric focusing the second dimension electrophoresis is run according to standard procedures.

After 2D electrophoresis, a spot map from respective gel is obtained by scanning the gel in a TYPHOON™ scanner at wavelengths specific for the various CY™ dyes. The spot maps are analysed using, for example the DECYDER™ software to ensure that there is no major protein loss in the fractionated samples compared to non-fractionated CY2™ labelled samples. Also how well the samples have been separated is analysed. Finally the proportions of overlapping areas of the spot maps from the different fractions are determined to estimate the performance of the pl fractionation.

### c) 2D Electrophoresis Using Narrow Range IPG Strips

When a particular protein or set of proteins with known pl's is/are of interest narrow range IPG strips as first dimension may be run following the above 2D run or directly after the pre-fractionation according to the invention. The protein samples may for example be run on one or several strips with pH ranges 3-5.6, 5.3-6.5, 6.2-7.5, 3.5-4.5, 4-5, 4.5-5.5, 5-6, 5.5-6.7, 3-7, 4-7, 7-11 or 6-9 depending on which fraction(s) of the sample is(are) of interest.

The 2D electrophoresis and analysis is done as described above.

Spots comprising proteins of interest may be picked from the gel for further analysis. According to the invention low abundant proteins will be more easily detected since pl fractionation will allow higher protein loads. This will result in better resolution and improved detection of low abundant/rare proteins.

The above examples illustrate specific aspects of the present invention and are not intended to limit the scope thereof in any respect and should not be so construed. Those skilled in the art having the benefit of the teachings of the present invention as set forth above, can effect numerous modifications thereto. These modifications are to be construed as being encompassed within the scope of the present invention as set forth in the appended claims.

1: A method for pre-fractionation of a complex protein and/or peptide sample, comprising the following steps:
a) loading the sample onto pre-packed media able to separate proteins/peptides according to their pI-value;
b) eluting the sample by centrifugal force under denaturing conditions with at least three buffers having different pH in a stepwise manner to obtain at least three fractions separated according to pI-value; and
c) subjecting each fraction to further separation in a narrow pH-range corresponding to the pI-values of said fractions.

2: The method of claim 1, wherein the flow through is collected before step b), wherein all fractions representing the total sample are obtained.

3: The method of claim 1, wherein the pre-packed media is chromatofocusing or ion exchange media.

4: The method of claim 1, wherein the ion exchange media in step a) is in a spin column or a multiwell plate format.

5: The method of claim 4, wherein the three buffers in step b) have pH values in the following intervals, pH 7-9, 6-4 and 3-2, respectively.

6: The method of claim 5, wherein the buffers have pH 8, 5 and 3, respectively.

7: The method of claim 4, wherein there are at least five buffers in step b) having pH 9, pH 7, pH 6, pH 5 and pH 3, respectively.

8: The method of claim 4, wherein the anion exchange column is filled with a weak anion exchanger.

9: The method of claim 1, wherein the further separation in step c) is 2D electrophoresis.

10: The method of claim 1, wherein the separation in step c) is electrophoretic separation on a narrow range IPG (immobilised pH gradient) strip or gel.

11: A kit comprising at least one spin column or multiwell plate filled with pre-packed chromatography media able to separate proteins/peptides according to pI-value, at least three buffers, and one or more IPG strips having a narrow pH-range adapted to each of said buffers.

12: The kit of claim 11, wherein the chromatography medium is an anion exchange or chromatofocusing medium and wherein the three buffers have pH values in the intervals pH 7-9, 6-4 and 3-2, respectively.

13: The kit of claim 11, wherein the three buffers are selected from 7M Urea, 2M Thiourea, 0.5% CHAPS™, 0.5% DTT and 0.5-2% ampholyte, tris pH 8 or polybuffer pH 5 or glycine pH 3.

14: The kit of claim 11, comprising at least five buffers.

15: The kit of claim 14, wherein the five buffers have pH 9, pH 7, pH 6, pH 5 and pH 3, respectively.

16: The kit of claim 15, wherein the five buffers are selected from 7M Urea, 2M Thiourea, 0.5% CHAPS™, 0.5% DTT and 0.5-2% ampholyte, tris pH 8 or tris pH 7 or polybuffer pH 6 or polybuffer pH 5 or glycine pH 3.

17: The kit of claim 11, wherein the IPG strips are pH 7-11, pH 6-9 and 3-5.6 NL further wherein the three buffers are selected from 7M Urea, 2M Thiourea, 0.5% CHAPS™ 0.5% DTT and 0.5-2% ampholyte, tris pH 8 or polybuffer pH 5 or glycine pH 3.

18: The kit of claim 11, wherein the IPG strips are pH 7-11 (for both elution fraction 1 and 2), pH 6-9, pH 5.3-6.5 and pH 3-5.6 NL, further wherein the five buffers are selected from 7M Urea, 2M Thiourea, 0.5% CHAPS™, 0.5% DTT and 0.5-2% ampholyte, tris pH 8 or tris pH 7 or polybuffer pH 6 or polybuffer pH 5 or glycine pH 3.

19: The kit of claim 11, further comprising at least two protein/peptide labelling reagents.
